(19) United States
(12) Patent Application Publication
(10) Pub. No.: US 2007/0111314 A1
(43) Pub. Date: May 17, 2007

(54) Methods for tagging and authenticating inks using compositions

(76) Inventors: Lufei Lin, San Diego, CA (US); John Helfrick, San Diego, CA (US); Sundar Vasudevan, Corvallis, OR (US); LeAnn Bell, Corvallis, OR (US); Mark G. Wisnosky, San Diego, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/269,093

(22) Filed: Nov. 8, 2005

(51) Int. Cl. G01N 33/24

(52) U.S. Cl. 436/27

(57) ABSTRACT

A method for tagging an ink with an isotopic tagging composition is provided. The method includes tagging the ink with a tagging composition having an artificial abundance of at least one isotope of an element to produce an authentic ink having an artificial abundance of the at least one isotope of the element which exceeds a natural abundance thereof in the authentic ink. A method for authenticating unidentified ink samples by comparing isotope abundances and/or abundance ratios in the unidentified ink samples with isotope abundances and/or abundance ratios in authentic inks tagged with isotopic tagging compositions is also provided. The method also includes tagging a pigment-based ink with trityl-protected thiol additives and methods for identifying inks containing the same.

### CROSS REFERENCE TO RELATED APPLICATION

This application is related to a patent application entitled, “Methods and Systems for Identifying Ink” (Attorney Docket No. 200316071-2), filed on even date with this application.

### FIELD OF THE INVENTION

The present invention relates to methods for identification. More specifically, the present invention relates to methods for tagging inks with isotopic tagging compositions or trityl-protected thiol additives. Further, the present invention relates to methods for authenticating unidentified ink samples.

### BACKGROUND OF THE INVENTION

Inkjet printers operate by placing extremely small droplets of ink onto a medium (e.g., a sheet of paper) to create an image. Inks used in inkjet printers are typically stored in and dispensed from one or more inkjet cartridges that are specific for the inkjet printer with which they are intended to be used. Once the ink in an inkjet cartridge has been used, the cartridge either must be replaced or refilled. Refilling of inkjet cartridges is a relatively simple task and refill kits are readily available.

While the ease with which inkjet cartridges may be refilled is a key component in the success that inkjet printers have enjoyed, it also lends itself to a high susceptibility for counterfeiting. This can lead, for instance, to damage to the reputation of an ink manufacturer if in an inkjet cartridge is replaced with a counterfeit ink of inferior quality and sold with the manufacturer's label still attached. Additionally, counterfeiting may lead to large expenditures of warranty monies paid out by an ink manufacturer if, for example, an authentic ink is replaced with a counterfeit ink, or diluted, and then returned to the manufacturer accompanied by a complaint of substandard quality. Accordingly, a reliable method for authenticating inkjet inks and for detecting counterfeit inks would be advantageous.

Techniques have been developed for tagging various articles to prevent counterfeiting, or at least reduce the incidence thereof. For instance, articles may be tagged with code-bearing micro-particles, bulk chemical substances, or radioactive substances. However, tagging techniques that are applicable to other articles or materials are not necessarily suitable for tagging inks. Inks are typically formulated to provide maximum performance in terms of, among other traits, color, physical and chemical properties, and interaction with the medium on which they are printed. Accordingly, a tagging substance for inks should have no effect on the attractiveness or performance of the ink. Additionally, an ideal tagging substance for inks should be inert at the concentration level used, should not contain any toxic components, and should be easily and reliably detected at trace concentration levels, safe to use, and inexpensive. Conventional tagging methods typically lack one or more of these characteristics.

Accordingly, a reliable method for tagging inks to reduce the incidence of counterfeiting which preserves the integrity and quality of the ink would be advantageous. Additionally, a method for tagging inks which cannot be easily duplicated and which is fast and inexpensive, yet provides a high level of security against counterfeiting, would be desirable.

### BRIEF SUMMARY OF THE INVENTION

The present invention provides a method for tagging an ink for identification. The method comprises tagging the ink with a tagging composition comprising an augmented abundance of at least one isotope of an element to produce an authentic ink having an artificial abundance of the at least one isotope of the element which exceeds a natural abundance thereof in the authentic ink. The at least one isotope may be present in the authentic ink in a concentration between 1 and 1000 parts per billion.

The present invention further provides a method for authenticating an unidentified ink comprising tagging an ink with a tagging composition having an augmented abundance of at least one isotope of an element to produce an authentic ink, the authentic ink having an artificial abundance of the at least one isotope which exceeds a natural abundance thereof in the authentic ink; obtaining a sample of the unidentified ink; detecting an abundance of the at least one isotope in the unidentified ink sample using inductively coupled plasma mass spectrometry; and determining whether the unidentified ink is the authentic ink based upon the detected abundance of the at least one isotope in the unidentified ink sample

Still further, the present invention provides a method for authenticating an ink. The method includes obtaining a sample of an unidentified ink and detecting an abundance of at least one isotope of an element in the unidentified ink sample using inductively coupled plasma mass spectrometry. The detected abundance is compared to a tagging record which correlates an authentic ink identifier with information regarding an authentic ink. The authenticity of the unidentified ink is determined based upon a comparison of the detected abundance of the at least one isotope in the unidentified ink sample and the tagging record.

Another embodiment of the present invention further provides a method for authenticating an unidentified ink comprising adding a composition having a trityl-protected thiol additive to a pigment-based ink. Acid is then added to the pigment-based ink to cause the pigment in the ink to coagulate. The coagulated pigment is then removed from the remaining ink components and the corresponding filtrate is assayed, which leads to the formation of a compound that can be quantitatively detected at a particular absorbance.

Other features and advantages of the present invention will become apparent to those of ordinary skill in the art through consideration of the ensuing description, the accompanying drawings and the appended claims.

### BRIEF DESCRIPTION OF THE SEVERAL VIEWS OF THE DRAWINGS

While the specification concludes with claims particularly pointing out and distinctly claiming that which is regarded as the present invention, the advantages of this invention may be more readily ascertained from the following description of the invention when read in conjunction with the accompanying drawings in which:

FIG. 1 shows an embodiment of an aspect of the present invention in a chart which illustrates the ⁶Li isotope count, ⁷Li isotope count, and ⁶Li/⁷Li isotope ratio of a number of authentic ink samples tagged with 10 ppb ⁶Li isotope and a corresponding number of ink samples which have been adulterated by 20% so that they contain 8 ppb ⁶Li;

FIG. 2 shows another embodiment of an aspect of the present invention in a chart which illustrates the ⁶Li isotope count, ⁷Li isotope count, and ⁶Li/⁷Li isotope ratio of a number of authentic ink samples tagged with 10 ppb ⁶Li isotope and a corresponding number of ink samples which have been adulterated by 50% so that they contain 5 ppb ⁶Li;

FIG. 3 shows yet another embodiment of an aspect of the present invention in a chart which illustrates the ⁶Li isotope count, ⁷Li isotope count, and ⁶Li/⁷Li isotope ratio of a number of authentic ink samples tagged with 10 ppb ⁶Li isotope and a corresponding number of ink samples which have been adulterated by 80% so that they contain 2 ppb ⁶Li;

FIG. 4 shows an additional embodiment of an aspect of the present invention in a chart which illustrates the ⁶Li isotope count, ⁷Li isotope count, and ⁶Li/⁷Li isotope ratio of a number of authentic ink samples tagged with 10 ppb ⁶Li isotope and a corresponding number of ink samples which have been diluted by 50% so that they contain 5 ppb ⁶Li; and

FIG. 5 shows a further embodiment of an aspect of the present invention in a chart which illustrates the ⁶Li isotope count, ⁷Li isotope count, and ⁶Li/⁷Li isotope ratio of number of authentic ink samples tagged with 10 ppb ⁶Li isotope and a corresponding number of ink samples which have been diluted by 80% so that they contain 2 ppb ⁶Li.

### DETAILED DESCRIPTION OF THE INVENTION

The present invention is directed to methods for tagging inks with isotopic tagging compositions and for authenticating inks or detecting counterfeit inks by comparing isotope abundances and/or abundance ratios in unidentified ink samples with isotope abundances and/or abundance ratios in authentic inks tagged with isotopic tagging compositions. The present invention is also directed to methods for tagging inks with trityl-protected thiol additives and for authenticating inks or detecting counterfeit inks tagged with such additives. The particular embodiments described herein are intended in all respects to be illustrative rather than restrictive. Other and further embodiments will become apparent to those of ordinary skill in the art to which the present invention pertains without departing from its scope.

In one embodiment, the present invention describes the use of tagging compositions for tagging or labeling an ink for identification and authentication thereof. As used herein, the term “tagging composition” or “labeling composition” refers to a composition having an augmented abundance of at least one isotope of an element. That is, the tagging compositions of the present invention are enhanced with one or more isotopes such that when incorporated into an ink, the ink comprises an artificial abundance of the at least one isotope which exceeds a natural abundance thereof. The tagging compositions thus may contain at least one isotope having an abundance that is detectably different from the natural amount of the at least one isotope in the tagging composition. As used herein, “detectably different” abundances means that the difference between the abundance of the tagging composition prior to incorporation of the at least one isotope and a respective abundance after incorporation of the at least one isotope is larger than the experimental error of the detection method utilized, e.g., inductively coupled plasma mass spectrometry (ICP-MS). The tagging compositions which may be used in the methods of the present invention may be non-toxic and inert at the desired concentration levels and are capable of incorporation into an ink without altering the attractiveness or performance thereof.

The tagging compositions of the present invention may also include a suitable carrier. Currently preferred carriers are materials in which one or more isotopes may be dispersed or dissolved. The carrier may be any material which is inert at the concentration level used (i.e., a material which has no reaction or limited reaction with the ink components) to facilitate the tagging compositions being incorporated into the inks to be tagged. In addition, the carrier should not adversely affect the detection of the isotopes in the inks. The carrier may be a liquid in which one or more isotopes may be dissolved to produce a substantially homogenous solution. Suitable liquids include, by way of example only, solvents, water, and alcohols.

The tagging compositions of the present invention may be incorporated into an ink to produce an authentic ink having the at least one isotope present in a trace amount (e.g., a concentration between 1 and 1000 parts per billion (ppb)). As used herein, the term “authentic ink” refers to an ink which has been tagged with a tagging composition of the present invention and which, accordingly, comprises an artificial abundance of at least one isotope of an element which exceeds a natural amount of the at least one isotope in the ink. By incorporating a tagging composition into an ink in this manner to produce an authentic ink, the quality and integrity of the ink may be preserved and yet the concentration of the at least one isotope in the ink may be detected by suitable detection technology. In a currently preferred embodiment, the detection technology may comprise inductively coupled plasma mass spectrometry (ICP-MS), which is capable of detecting the isotope concentration for a number of different isotopes in the parts per billion (ppb) range in a relatively short period of time, (e.g., less than 10 minutes).

The present invention provides a method for tagging an ink for identification. In one embodiment, the method comprises incorporating into an ink, a tagging composition which includes an augmented abundance of at least one isotope of an element to produce an authentic ink. Isotopes may be prepared or isolated for use in the tagging compositions using conventional isotope extraction methods, including plasma separation processes, electromagnetic separation, molecular laser isotope separation, atomic vapor laser isotope separation, gas centrifugation, gas diffusion, and distillation. Each of these methods is well known to those of ordinary skill in the art and, accordingly, isotope extraction is not further discussed herein. Additionally, highly enriched samples of most stable isotopes are commercially available from a number of sources including, but not limited to, Inorganic Ventures/IV Labs of Lakewood, N.J.

Any element having at least one stable isotope may be used in the tagging compositions of the present invention. However, light elements are currently preferred to heavy elements, and elements of the alkali group are currently preferred as they tend to have limited interaction with the other components which typically are included in inkjet inks, as well as having limited interaction with the inkjet cartridge components. Further, if certain transition and/or heavy metals are utilized, precipitates may be formed which can result in ink performance failure.

By way of example, and not limitation, elements which are currently preferred for use in the tagging compositions of the present invention, alone or in combination, include lithium (Li), rubidium (Rb), cesium (Cs), certain alkaline metals (e.g., beryllium (Be), magnesium (Mg), strontium (Sr), and barium (Ba)), certain transition metals (e.g., manganese (Mn), cobalt (Co), nickel (Ni), copper (Cu), zinc (Zn), yttrium (Y), niobium (Nb), rhodium (Rh), and rhenium (Re)), and certain rare earth elements (e.g., lanthanum (La), cerium (Ce), praseodymium (Pr), europium (Eu), gadolinium (Gd), terbium (Tb), and lutetium (Lu)).

Each of the above listed exemplary elements has at least one stable isotope. However, not all isotopes of each of the elements are currently preferred. In this regard, isotopes of the exemplary elements which are currently preferred for use in the tagging compositions of the present invention include, by way of example only and not limitation, ⁶Li, ⁷Li, ⁸⁵Rb, ⁸⁷Rb, ¹³³Cs, ⁹Be, ²⁴Mg, ²⁵Mg, ²⁶Mg, ⁸⁴Sr, ⁸⁶Sr, ⁸⁷Sr, ⁸⁸Sr, ¹³⁰Ba, ¹³²Ba, ¹³⁴Ba, ¹³⁵Ba, ¹³⁶Ba, ¹³⁷Ba, ¹³⁸Ba, ⁵⁵Mn, ⁵⁹Co, ⁵⁸Ni, ⁶⁰Ni, ⁶²Ni; ⁶³Cu, ⁶⁵C ⁶⁴Zn, ⁶⁶Zn ⁶⁸Zn, ⁹³Nb, ¹⁰³Rh, ¹⁸⁵Re, ¹⁸⁷Re, ¹³⁹La, ¹⁴⁰Ce, ¹⁴¹Pr, ¹⁵¹Eu, ⁵³Eu, ⁵²Gd, ⁵⁴Gd, ¹⁵⁵Gd, ¹⁵⁷Gd, ¹⁶⁰Gd, ¹⁵⁹Tb, and ¹⁷⁵Lu.

In a particular embodiment, the ⁶Li isotope is currently preferred as it is unique and highly sensitive to detection via ICP-MS in the shield plate cool plasma condition, i.e., it can be detected in a concentration as low as between 1 and 100 ppb. The element lithium (Li) has two stable isotopes with atomic masses of 6 and 7. In natural lithium, these two isotopes are present in a concentration of 7.50% and 92.50%, respectively. Accordingly, when an ink is tagged with the ⁶Li isotope, the abundance of the ⁷Li isotope is altered as well.

Combinations of isotopes and elements may be incorporated into the tagging compositions of the present invention to create authentic inks that are extremely difficult for would be counterfeiters to replicate.

Once a tagging composition of the present invention has been incorporated into an ink to produce an authentic ink, the authentic ink may have an artificial abundance of the at least one isotope that was present in the tagging composition which exceeds a natural abundance thereof. Further, the at least one isotope may be present in the authentic ink in a concentration suitable for detection using ICP-MS (e.g., between about 1 and about 1000 parts per billion (ppb). More particularly, if the ⁶Li isotope is selected as the at least one isotope in the tagging composition, the concentration of the ⁶Li isotope in the authentic ink may be detectable in a range of between about 1 and about 100 parts per billion. If an isotope of Be, Mg, or Rb is selected as the at least one isotope, the isotope concentration in the authentic ink may be detectable in a range of between about 10 and about 1000 ppb. If an isotope of a rare earth element is selected as the at least one isotope, the isotope concentration in the authentic ink may be detectable in a range of between about 10 and about 500 ppb.

In another embodiment, the tagging compositions of the present invention may comprise an augmented abundance of at least one isotope of a first element and an augmented abundance of at least one isotope of a second element, the first and second elements being different from one another. Once a tagging composition in accordance with this embodiment is incorporated into an ink to produce an authentic ink, the authentic ink may have an artificial abundance of the at least one isotope of the first element and an artificial abundance of the at least one isotope of the second element, each of which exceeds a respective natural abundance thereof in the authentic ink. It is currently preferred that the at least one isotope of each of the first and second elements be present in the authentic ink in a concentration between about 1 and about 1000 parts per billion.

In this instance, the intensity responses as measured by ICP-MS may be independent; however, the ratio of the mass intensities may remain constant. Accordingly, a counterfeit ink may still be detected using ICP-MS if none of either of the isotopes is artificially introduced into the counterfeit ink. Elements and isotopes as set forth above may be utilized for each of the at least one isotope of the first element and the at least one isotope of the second element.

In yet another embodiment, the tagging compositions of the present invention may comprise an augmented abundance of at least two isotopes of a single element. Once a tagging composition in accordance with this embodiment is incorporated into an ink to produce an authentic ink, the authentic ink may have an artificial abundance of each of the at least two isotopes of the element, which exceeds a respective natural abundance thereof in the authentic ink. Additionally, an authentic isotope ratio, (i.e., the ratio of the artificial abundance of a first of the at least two isotopes) to the artificial abundance of a second of the at least two isotopes, may be different from a natural abundance ratio of the at least two isotopes of the element in the authentic ink (that has not been tagged). It is currently preferred that each of the at least two isotopes of the element be present in the authentic ink in a concentration between 1 and 1000 parts per billion. Elements and isotopes as set forth above may be utilized for each of the at least two isotopes in this embodiment.

The present invention also provides a method for authenticating an unidentified ink. The method comprises comparing information extracted from a sample of the unidentified ink with information that is known about an authentic ink and the one or more tagging compositions with which the authentic ink has been tagged, to determine whether the unidentified ink sample is a sample of the authentic ink or is a sample of a counterfeit ink. Authentic inks for use in the method of the present invention may be prepared using the elements and isotopes as previously set forth. In one embodiment, the method includes obtaining a sample of an unidentified ink and detecting an abundance in the unidentified ink sample of the at least one isotope with which the authentic ink has been tagged using ICP-MS. The detected abundance of the at least one isotope in the unidentified ink sample may subsequently be compared with the known artificial abundance of the at least one isotope in the authentic ink to determine whether the unidentified ink sample is the authentic ink.

As the natural abundance of a given isotope in an ink is not constant, a simple comparison of the raw abundance numbers (i.e., isotope mass counts) will generally not provide an accurate indication of whether or not the two inks being compared are the same ink. Accordingly, in one embodiment, it is currently preferred that a ratio of the detected abundance of the at least one isotope in the unidentified ink sample to the artificial abundance of the at least one isotope in the authentic ink be determined. If this ratio is less than 0.66, the unidentified ink sample may be designated as counterfeit. However, if this ratio is greater than or equal to 0.66, it is likely that the unidentified ink sample is a sample of the authentic ink.

In accordance with the method of the present invention, an ink may be tagged with a tagging composition comprising an augmented abundance of at least one isotope of a first element and an augmented abundance of at least one isotope of a second element, the first and second elements being different from one another, to produce the authentic ink. If a tagging composition in accordance with this embodiment is utilized to produce the authentic ink, it is currently preferred that an abundance of each of the at least one isotope of the first element and the at least one isotope of the second isotope be detected in the unidentified ink sample and that information regarding the artificial abundance of the at least one isotope of each of the first and second elements be own with respect to the authentic ink. Using this information, a ratio of the detected abundance of the at least one isotope of the first element in the unidentified ink sample to the artificial abundance of the at least one isotope of the first element in the authentic ink may be determined, as may a ratio of the detected abundance of the at least one isotope of the second element in the unidentified ink sample to the artificial abundance of the at least one isotope of the second element in the authentic ink. If either or both of these ratios are less than 0.66, the unidentified ink sample may be designated as counterfeit.

Additionally, an unidentified isotope ratio may be determined for the unidentified ink sample. The unidentified isotope ratio is a ratio of the detected abundance of the at least one isotope of the first element to the detected abundance of the at least one isotope of the second element in the unidentified ink sample. Similarly, an authentic isotope ratio, i.e., the ratio of the artificial abundance of the at least one isotope of the first element to the artificial abundance of the at least one isotope of the second element in the authentic ink sample, may be determined. If the ratio of the unidentified isotope ratio to the authentic isotope ratio is less than 0.66, the unidentified ink sample may be designated as counterfeit.

Further, in accordance with the method of the present invention, an ink may be tagged with a tagging composition comprising an augmented abundance of at least two isotopes of a single element to produce an authentic ink. If a tagging composition in accordance with this embodiment is utilized to produce the authentic ink, it is currently preferred that an abundance of each of the at least two isotopes of the element be detected in the unidentified ink sample and that information regarding the artificial abundance of each of the at least two isotopes of the element be known with respect to the authentic ink. It is also currently preferred that an unidentified isotope ratio, (i.e., the ratio of the detected abundance of a first of the at least two isotopes of the element to the detected abundance of a second of the at least two isotopes of the element) be determined for the unidentified ink sample and that an authentic isotope ratio (i.e., the ratio of the artificial abundance of a first of the at least two isotopes of the element to the artificial abundance of a second of the at least two isotopes of the element) be determined for the authentic ink.

Using this information, a ratio of the detected abundance of a first of the at least two isotopes of the element in the unidentified ink sample to the artificial abundance of the first of the at least two isotopes of the element in the authentic ink may be determined, as may a ratio of the detected abundance of the second of the at least two isotopes of the element in the unidentified ink sample to the artificial abundance of the second of the at least two isotopes of the element in the authentic ink. If either or both of these ratios are less than 0.66, the unidentified ink sample may be designated as counterfeit. Further, if the ratio of the unidentified isotope ratio to the authentic isotope ratio is less than 0.66, the unidentified ink sample may also be designated as counterfeit.

To facilitate a comparison between information extracted from an unidentified ink sample and information known about an authentic ink, information regarding the authentic ink may be recorded in a tagging record for use in determining authenticity and detecting counterfeit inks. The term “tagging record,” as used herein, refers to information (record) which correlates an identifier for the authentic ink with information regarding the isotope(s) with which the ink has been tagged. For instance, an authentic ink identifier may comprise one or more pieces of information about the ink including, but not limited to, a serial number or batch number, the date of manufacture, and the name brand of the ink. The authentic ink identifier may be correlated in the tagging record with information regarding the isotope(s) with which the ink has been tagged, including, but not limited to, the element used for tagging, the isotope of the element that was used (if there is more than one stable isotope), and the concentration at which the isotope is present in the ink. It is currently preferred that tagging records are made at the time an authentic ink is tagged.

In yet another embodiment, the present invention provides a method for authenticating an ink an unidentified ink comprising adding a tagging composition having a triphenylmethyl(trityl) protected thiol additive to a pigment-based ink. Acid is then added to the pigment-based ink in sufficient amount to cause the pigment in the ink to coagulate. The trityl protecting groups are also removed from the thiol. The coagulated pigment is then removed from the remaining ink components by any suitable means, such as filtration or centrifugation. The corresponding filtrate or supernate is then analyzed by any suitable means, such as an Ellman assay. For example, where S-tritylcysteine is added to a pigment-based ink, 5,5′-dithiobis(2-nitrobenzoic acid) can be added to the filtrate (post acid addition and filtration). This leads to the formation of a 5-thio-2-nitrobenzoic acid, which can be quantitatively detected by its absorbance at 512 nm.

The following examples describe methods for authenticating an ink in accordance with the present invention. The examples utilize an ink tagged with an artificial abundance of lithium isotope ⁶Li (and, accordingly, an artificial abundance of lithium isotope ⁷Li as well). Other examples utilize an ink tagged with a trityl-protected thiol additive. It will be understood and appreciated by those of ordinary skill in the art, however, that the methods illustrated are useful with other isotopes and other elements as well. The examples are merely illustrative and are not meant to limit the scope of the present invention in any way.

### EXAMPLES

### Example 1

### Preparation of an Authentic Ink

An isotope solution enriched with the ⁶Li isotope was obtained from Inorganic Ventures/IV Labs of Lakewood, N.J. The ⁶Li isotope enriched solution contained about 1002±1 ppm ⁶Li and 67±1 ppm ⁷Li in 5.0% HNO₃. Thus, it can be seen that the ⁶Li isotope enriched solution contained about 6.3% ⁷Li isotope as well. A 5 part per million (ppm) ⁶Li isotope stock solution was prepared from the ⁶Li enriched isotope solution by diluting the ⁶Li enriched isotope solution with deionized water. A tagged ink was subsequently prepared from the ⁶Li isotope stock solution by adding 0.1 g of the ⁶Li isotope stock solution to 50 g of ink. The resulting ink was a 10 ppb ⁶Li isotope tagged ink which was labeled as the “authentic” ink.

Subsequently, a sample of the authentic ink was diluted 100 fold with deionized water. The diluted samples were then introduced into an Agilent-4500 Inductively Coupled Plasma Mass Spectrometry (ICP-MS) instrument, obtained from Agilent Technologies of Palo Alto, Calif. and the cool plasma method was used to measure the isotope mass counts. The ICP-MS instrumentation and the cool plasma method are well known to those of ordinary skill in the art and, accordingly, are not discussed further herein. Cobalt (Co) was added as the internal standard to compensate for any instrument drift and sample matrix effect. The instrument was not tuned to optimize the detection of light masses.

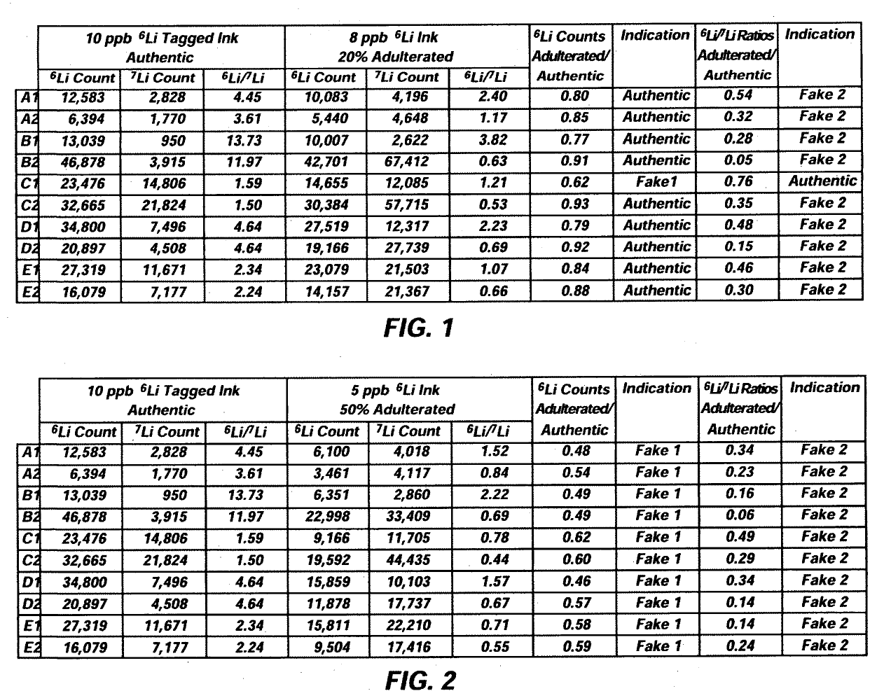

⁶Li isotope and ⁷Li isotope mass counts, as well, as the ratio of ⁶Li:⁷Li are shown in each of FIGS. 1-5 for the authentic ink. The authentic ink is labeled as 10 ppb ⁶Li Tagged Ink.

A number of different inks were tagged in this manner and are labeled as ink samples A-E in FIGS. 1-5. Ink sample A was a black ink, ink sample B was a magenta ink, ink sample C was a yellow ink, ink sample D was a cyan ink having a first formulation, and ink sample E was a cyan ink having a second formulation. Each of the ink samples was prepared for purposes of the experiments at the San Diego, Calif. facility of Hewlett-Packard Company. Additionally, a number of the below-described experiments were run at two separate times. Accordingly, each of ink samples A-E is also labeled as 1 and 2 to indicate two different runs.

### Example 2

### Preparation of 20% Adulterated Ink

An ink adulterated by 20% relative to the authentic ink was prepared by mixing a sample of the authentic ink with an untagged ink of the same type and color to obtain an 8 ppb ⁶Li adulterated ink. Subsequently, samples of the 20% adulterated ink were diluted 100 fold with deionized water. The diluted samples were then introduced into the Agilent-4500 ICP-MS instrument and the cool plasma method was used to measure the isotope mass counts. As with the authentic ink samples, cobalt (Co) was added as the internal standard to compensate of any instrument drift and sample matrix effect and the instrument was not tuned to optimize the detection of light masses.

The ⁶Li and ⁷Li mass counts, as well as the ratio of ⁶Li:⁷Li are shown in FIG. 1. The 20% adulterated ink is labeled as 8 ppb ⁶Li Ink.

In a first comparison, using the ⁶Li mass counts of both the authentic ink and the 20% adulterated ink, the ratio of the ⁶Li mass count of the adulterated ink to the ⁶Li mass count of the authentic ink was determined. These values are shown for each ink sample in FIG. 1 under the heading ⁶Li Counts Adulterated/Authentic.

If the ratio of the ⁶Li mass count of the adulterated ink to the ⁶Li mass count of the authentic ink was greater than 0.66, the ink sample was indicated as “Authentic.” If, however, the ratio of the ⁶Li mass count of the adulterated ink to the ⁶Li mass count of the authentic ink was less than or equal to 0.66, the ink sample was indicated as “Fake 1.” As shown in FIG. 1, each of samples A1, A2, B1, B2, C2, D1, D2, E1, and E2 was indicated as Authentic and sample C1 was indicated as “Fake 1.”

In a second comparison, using the ⁶Li:⁷Li ratios for both the authentic ink and the 20% adulterated ink, the ratio of the ⁶Li:⁷Li ratio of the adulterated ink to the ⁶Li:⁷Li ratio of the authentic ink was determined. These values are also shown for each ink sample in FIG. 1 under the heading ⁶Li/⁷Li Ratios Adulterated/Authentic.

If the ⁶Li:⁷Li ratio of the adulterated ink to the ⁶Li ⁷Li ratio of the authentic ink was greater than 0.66, the ink sample was indicated as “Authentic.” If, however, the ⁶Li:⁷Li ratio of the authentic ink was less than or equal to 0.66, the ink sample was indicated as “Fake 2.” As shown in FIG. 1, each of samples A1, A2, B1, B2, C2, D1, D2, E1, and E2 was indicated as “Fake 2” and ink sample C1 was indicated as “Authentic.”

If either of the first or second comparisons yielded an ink that was indicated as fake (i.e., either “Fake 1” or “Fake 2”), the ink was designated as counterfeit. As such, at only 20% adulteration, the methods of the present invention identified each of the ink samples as counterfeit.

### Example 3

### Preparation of 50% Adulterated Ink

An ink adulterated by 50% relative to the authentic ink was prepared by mixing a sample of the authentic ink with an untagged ink of the same type and color to obtain a 5 ppb ⁶Li adulterated ink. Subsequently, samples of the 50% adulterated ink were diluted 100 fold with deionized water. The diluted samples were then introduced into the Agilent-4500 ICP-MS instrument and the cool plasma method was used to measure the isotope mass counts. As with the authentic ink samples, cobalt (Co) was added as the internal standard to compensate for any instrument drift and sample matrix effect and the instrument was not tuned to optimize the detection of light masses.

The ⁶Li and ⁷Li mass counts, as well as the ratio of ⁶Li:⁷Li are shown in FIG. 2. The 50% adulterated ink is labeled as 5 ppb ⁶Li Ink.

In a first comparison, using the ⁶Li mass counts of both the authentic ink and the 50% adulterated ink, the ratio of the ⁶Li mass count of the adulterated ink to the ⁶Li mass count of the authentic ink was determined. These values are shown for each ink sample in FIG. 2 under the heading ⁶Li Counts Adulterated/Authentic.

If the ratio of the ⁶Li mass count of the adulterated ink to the ⁶Li mass count of the authentic ink was greater than 0.66, the ink sample was indicated as “Authentic.” If, however, the ratio of the ⁶Li mass count of the adulterated ink to the ⁶Li mass count of the authentic ink was less than or equal to 0.66, the ink sample was indicated as “Fake 1.” As shown in FIG. 2, each of the ink samples was indicated as “Fake 1.”

In a second comparison, using the ⁶Li:⁷Li ratios for both the authentic ink and the 50% adulterated ink, the ratio of the ⁶Li:⁷Li ratio of the adulterated ink to the ⁶Li:⁷Li ratio of the authentic ink was determined. These values are also shown for each ink sample in FIG. 2 under the heading ⁶Li/⁷Li Ratios Adulterated/Authentic.

If the ⁶Li:⁷Li ratio of the adulterated ink to the ⁶Li:⁷Li ratio of the authentic ink was greater than 0.66, the ink sample was indicated as “Authentic.” If, however, the ⁶Li:⁷Li ratio of the authentic ink was less than or equal to 0.66, the ink sample was indicated as “Fake 2.” As shown in FIG. 2, each of the ink samples in this experiment was indicated as “Fake 2.”

If either of the first or second comparisons yielded an ink that was indicated as fake (i.e., either “Fake 1” or “Fake 2”), the ink was designated as counterfeit. As such, at 50% adulteration, the methods of the present invention identified each of the ink samples as counterfeit.

### Example 4

### Preparation of 80% Adulterated Ink

An ink adulterated by 80% relative to the authentic ink was prepared by mixing a sample of the authentic ink with an untagged ink of the same type and color to obtain an 2 ppb ⁶Li adulterated ink. Subsequently, samples of the 80% adulterated ink were diluted 100 fold with deionized water. The diluted samples were then introduced into the Agilent-4500 ICP-MS instrument and the cool plasma method was used to measure the isotope mass counts. As with the authentic ink samples, cobalt (Co) was added as the internal standard to compensate for any instrument drift and sample matrix effect and the instrument was not tuned to optimize the detection of light masses.

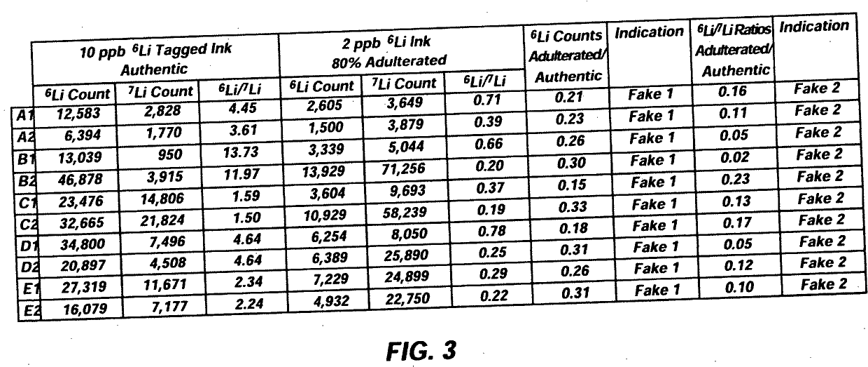

The ⁶Li and ⁷Li mass counts, as well as the ratio of ⁶Li:⁷Li are shown in FIG. 3. The 80% adulterated ink is labeled as 2 ppb ⁶Li Ink.

In a first comparison, using the ⁶Li mass counts of both the authentic ink and the 80% adulterated ink, the ratio of the ⁶Li mass count of the adulterated ink to the ⁶Li mass count of the authentic ink was determined. These values are shown for each ink sample in FIG. 3 under the heading ⁶Li Counts Adulterated/Authentic.

If the ratio of the ⁶Li mass count of the adulterated ink to the ⁶Li mass count of the authentic ink was greater than 0.66, the ink sample was indicated as “Authentic.” If, however, the ratio of the ⁶Li mass count of the adulterated ink to the ⁶Li mass count of the authentic ink was less than or equal to 0.66, the ink sample was indicated as “Fake 1.” As shown in FIG. 3, each of the ink samples was indicated as “Fake 1.”

In a second comparison, using the ⁶Li:⁷Li ratios for both the authentic ink and the 80% adulterated ink, the ratio of the ⁶Li:⁷Li ratio of the adulterated ink to the ⁶Li:⁷Li ratio of the authentic ink was determined. These values are also shown for each ink sample in FIG. 3 under the heading ⁶Li/⁷Li Ratios Adulterated/Authentic.

If the ⁶Li:⁷Li ratio of the adulterated ink to the ⁶Li:⁷Li ratio of the authentic ink was greater than 0.66, the ink sample was indicated as “Authentic.” If, however, the ⁶Li:⁷Li ratio of the authentic ink was less than or equal to 0.66, the ink sample was indicated as “Fake 2.” As shown in FIG. 3, each of the ink samples was indicated as “Fake 2.”

If either of the first or second comparisons yielded an ink that was indicated as fake (i.e., either “Fake 1” or “Fake 2”), the ink was designated as counterfeit. As such, at 80% adulteration, the methods of the present invention identified each of the ink samples as counterfeit.

### Example 5

### Preparation 50% Diluted Ink

An ink diluted by 50% relative to the authentic ink with deionized water was prepared to obtain an 5 ppb ⁶Li diluted ink. Subsequently, the 50% diluted ink sample was diluted 100 fold with deionized water. The diluted sample was then introduced into the Agilent-4500 ICP-MS instrument and the cool plasma method was used to measure the isotope mass counts. As with the authentic ink samples, cobalt (Co) was added as the internal standard to compensate for any instrument drift and sample matrix effect and the instrument was not tuned to optimize the detection of light masses.

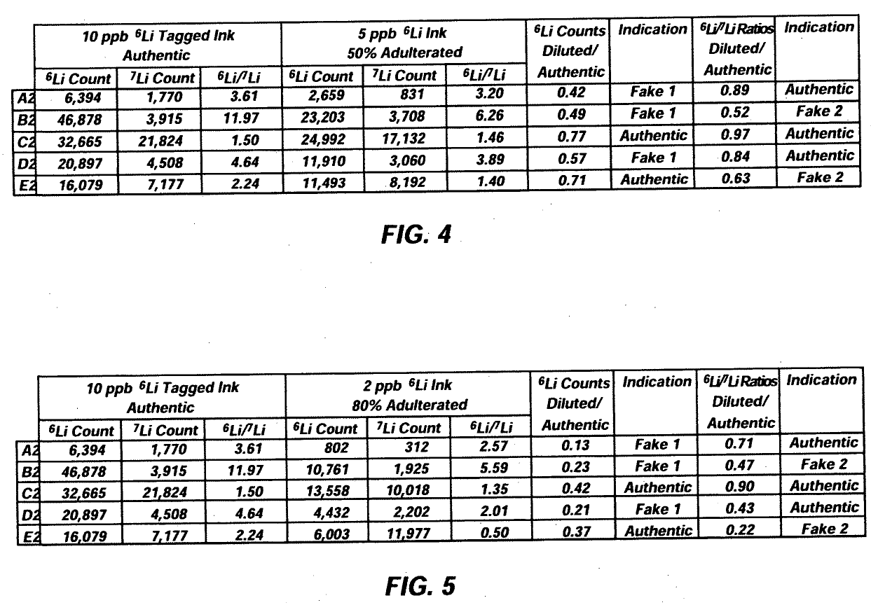

The ⁶Li and ⁷Li mass counts, as well as the ratio of ⁶Li:⁷Li are shown in FIG. 4. The 50% diluted ink is labeled as 5 ppb ⁶Li Ink.

In a first comparison, using the ⁶Li mass counts of both the authentic ink and the 50% diluted ink, the ratio of the ⁶Li mass count of the adulterated ink to the ⁶Li mass count of the authentic ink was determined. These values are shown for each ink sample in FIG. 4 under the heading ⁶Li Counts Adulterated/Authentic.

If the ratio of the ⁶Li mass count of the adulterated ink to the ⁶Li mass count of the authentic ink was greater than 0.66, the ink sample was indicated as “Authentic.” If, however, the ratio of the ⁶Li mass count of the adulterated ink to the ⁶Li mass count of the authentic ink was less than or equal to 0.66, the ink sample was indicated as “Fake 1.” As shown in FIG. 4, each of samples A2, B2 and D2 was indicated as “Fake 1” and each of samples C2 and E2 was indicated as “Authentic.”

In a second comparison, using the ⁶Li:⁷Li ratios for both the authentic ink and the 50% diluted ink, the ratio of the ⁶Li:⁷Li ratio of the adulterated ink to the ⁶Li:⁷Li ratio of the authentic ink was determined. These values are also shown for each ink sample in FIG. 4 under the heading ⁶Li/⁷Li Ratios Adulterated/Authentic.

If the ⁶Li:⁷Li ratio of the adulterated ink to the ⁶Li:⁷Li ratio of the authentic ink was greater than 0.66, the ink sample was indicated as “Authentic.” If, however, the ⁶Li:⁷Li ratio of the authentic ink was less than or equal to 0.66, the ink sample was indicated as “Fake 2.” As shown in FIG. 4, each of samples A2, C2, and D2 was indicated as “Authentic” and each of samples B2 and E2 was indicated as “Fake 2.”

If either of the first or second comparisons yielded an ink that was indicated as fake (i.e., either “Fake 1” or “Fake 2”), the ink was designated as counterfeit. As such, at 50% dilution, the methods of the present invention identified four out of the five ink samples as counterfeit. Ink sample C2 was indicated as “Authentic” in both comparisons indicating possible contamination or detection error.

### Example 6

### Preparation of 80% Diluted Ink

An ink diluted by 80% relative to the authentic ink with deionized water was prepared to obtain an 2 ppb ⁶Li diluted ink. Subsequently, the 80% diluted ink sample was further diluted 100 fold with deionized water. The diluted sample was then introduced into the Agilent-4500 ICP-MS instrument and the cool plasma method was used to measure the isotope mass counts. As with the authentic ink samples, cobalt (Co) was added as the internal standard to compensate for any instrument drift and sample matrix effect and the instrument was not tuned to optimize the detection of light masses.

The ⁶Li and ⁷Li mass counts, as well as the ratio of ⁶Li:⁷Li are shown in FIG. 5. The 80% diluted ink is labeled as 2 ppb ⁶Li Ink.

In a first comparison, using the ⁶Li mass counts of both the authentic ink and the 80% diluted ink, the ratio of the ⁶Li mass count of the adulterated ink to the ⁶Li mass count of the authentic ink was determined. These values are shown for each ink sample in FIG. 5 under the heading ⁶Li Counts Adulterated/Authentic.

If the ratio of the ⁶Li mass count of the adulterated ink to the ⁶Li mass count of the authentic ink was greater than 0.66, the ink sample was indicated as “Authentic.” If, however, the ratio of the ⁶Li mass count of the adulterated ink to the ⁶Li mass, count of the authentic ink was less than or equal to 0.66, the ink sample was indicated as “Fake 1.” As shown in FIG. 5, each of the ink samples was indicated as “Fake 1.”

In a second comparison, using the ⁶Li:⁷Li ratios for both the authentic ink and the 80% diluted ink, the ratio of the ⁶Li:⁷Li ratio of the adulterated ink to the ⁶Li:⁷Li ratio of the authentic ink was determined. These values are also shown for each ink sample in FIG. 5 under the heading ⁶Li/⁷Li Ratios Adulterated/Authentic.

If the ⁶Li:⁷ Li ratio of the adulterated ink to the ⁶Li:⁷Li ratio of the authentic ink was greater than 0.66, the ink sample was indicated as “Authentic.” If, however, the ⁶Li:⁷Li ratio of the authentic ink was less than or equal to 0.66, the ink sample was indicated as “Fake 2.” As shown in FIG. 5, each of samples B2, D2, and E2 was indicated as “Fake 2” and each of ink samples A2 and C2 was indicated as “Authentic.”

If either of the first or second comparisons yielded an ink that was indicated as fake (i.e., either “Fake 1” or “Fake 2”), the ink was designated as counterfeit. As such, at 80% dilution, the methods of the present invention identified each of the ink samples as counterfeit.

### Example 7

S-tritylcysteine is added to a pigment-based ink. The reactions involved in detection of the ink having the S-tritylcysteine additive are described as follows: 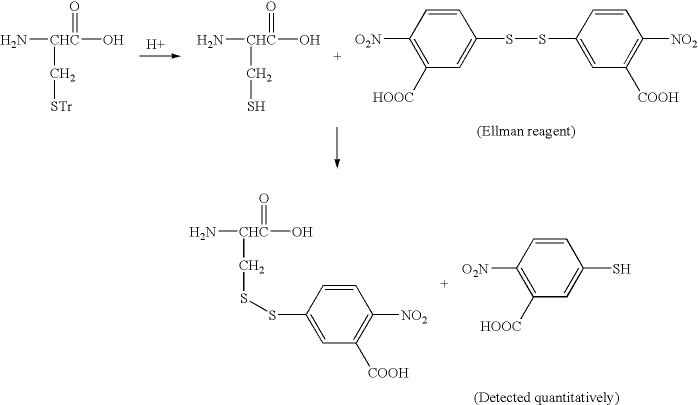
Addition of acid to pigment based ink containing this additive would accomplish two things. It would deprotect the trityl group from the compound as well as lead to coagulation of pigment dispersion. Thus, after addition of acid, the coagulated ink is filtered or centrifuged and the filtrate or supernate is added to an Ellman reagent. This would lead to the formation of the aromatic species shown above, which can be quantitatively detected by its absorbance at 512 nm. This is a quantitative method to detect cysteine that is well known to persons having skill in the art and which is widely used in biochemistry. While the present example has been described through use of an S-tritylcysteine additive, it is understood that any of its derivatives, such as the corresponding amide or methyl ester, could also be employed in the present invention.

Although the foregoing description contains many specifics, these should not be construed as limiting the scope of the present invention, but merely as providing illustrations of some exemplary embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions and modifications to the invention, as disclosed herein, which fall within the meaning and scope of the claims, are to be embraced thereby.

What is claimed is:

1. A method for tagging an ink for identification, comprising:
providing the ink; and
mixing the ink with a tagging composition comprising an augmented abundance of at least one isotope of an element to produce an authentic ink, the authentic ink having an artificial abundance of at least one isotope of the element which exceeds a natural abundance of the at least one isotope in the authentic ink, the at least one isotope being present in the authentic ink in a concentration between 1 and 1000 parts per billion.

2. The method of claim 1, wherein mixing the ink with a tagging composition comprises mixing the ink with a tagging composition comprising an augmented abundance of at least one isotope of an element selected from the group consisting of lithium, rubidium, and cesium.

3. The method of claim 2, wherein mixing the ink with a tagging composition comprising an augmented abundance of at least one isotope of an element to produce an authentic ink comprises mixing the ink with a tagging composition comprising an augmented abundance of at least one isotope selected from the group consisting of ⁶lithium, ⁷lithium, ⁸⁵rubidium, ⁸⁷rubidium, and ¹³³cesium.

4. The method of claim 3, wherein mixing the ink with a tagging composition comprising an augmented abundance of at least one isotope of an element to produce an authentic ink comprises mixing the ink with a tagging composition comprising an augmented abundance of the ⁶lithium isotope, wherein the ⁶lithium isotope is present in the authentic ink in a concentration between 1 and 100 parts per billion.

5. The method of claim 1, wherein mixing the ink with a tagging composition comprising an augmented abundance of at least one isotope of an element to produce an authentic ink comprises mixing the ink with a tagging composition comprising an augmented abundance of at least one isotope of an alkaline metal selected from the group consisting of beryllium, magnesium, strontium, and barium.

6. The method of claim 1, wherein mixing the ink with a tagging composition comprising an augmented abundance of at least one isotope of an element to produce an authentic ink comprises mixing the ink with a tagging composition comprising an augmented abundance of at least one isotope of rubidium, beryllium or magnesium, and wherein the rubidium, beryllium or magnesium isotope is present in the authentic ink in a concentration between 10 and 1000 parts per billion.

7. The method of claim 1, wherein mixing the ink with a tagging composition comprising an augmented abundance of at least one isotope of an element to produce an authentic ink comprises mixing the ink with a tagging composition comprising an augmented abundance of at least one isotope of a transition metal selected from the group consisting of manganese, cobalt, nickel, copper, zinc, yttrium, niobium, rhodium, and rhenium.

8. The method of claim 1, wherein mixing the ink with a tagging composition comprising an augmented abundance of at least one isotope of a transition metal to produce an authentic ink comprises mixing the ink with a tagging composition comprising an augmented abundance of at least one isotope of a rare earth element selected from the group consisting of lanthanum, cerium, praseodymium, europium, gadolinium, terbium, and lutetium.

9. The method of claim 1, wherein mixing the ink with a tagging composition comprising an augmented abundance of at least one isotope of an element comprises mixing the ink with a tagging composition comprising an augmented abundance of at least one isotope of a first element and at least one isotope of a second element, the first and second elements being different from one another, to produce an authentic ink having an artificial abundance of the at least one isotope of the first element and the at least one isotope of the second element each of which exceeds a respective natural abundance thereof in the authentic ink, and wherein the at least one isotope of each of the first and second elements is present in the authentic ink in a concentration between 1 and 1000 parts per billion.

10. The method of claim 1, wherein mixing the ink with a tagging composition comprising an augmented abundance of at least one isotope of an element to produce an authentic ink comprises mixing the ink with a tagging composition comprising an augmented abundance of at least two isotopes of an element, wherein the artificial abundance of each of the at least two isotopes exceeds a respective natural abundance thereof in the authentic ink, wherein an isotope ratio of the respective artificial abundances of the at least two isotopes is different from a natural abundance ratio of the at least two isotopes in the authentic ink, and wherein each of the at least two isotopes is present in the authentic ink in a concentration between 1 and 1000 parts per billion.

11. A method for authenticating an unidentified ink, comprising:
tagging an ink with a tagging composition comprising an augmented abundance of at least one isotope of an element to produce an authentic ink, the authentic ink having an artificial abundance of the at least one isotope of an element which exceeds a natural abundance of the at least one isotope in the authentic ink, the at least one isotope being present in the authentic ink in a concentration between 1 and 1000 parts per billion;
obtaining a sample of the unidentified ink;
detecting an abundance of the at least one isotope in the unidentified ink sample using inductively coupled plasma mass spectrometry; and
determining whether the unidentified ink is the authentic ink based upon the detected abundance of the at least one isotope in the unidentified ink sample.

12. The method of claim 11, wherein determining whether the unidentified ink is the authentic ink comprises determining a ratio of the detected abundance of the at least one isotope in the unidentified ink sample to the artificial abundance of the at least one isotope in the authentic ink, wherein if the ratio of the detected abundance to the artificial abundance is less than 0.66, the unidentified ink is designated as counterfeit.

13. The method of claim 11, wherein tagging an ink with a tagging composition comprises tagging an ink with a tagging composition comprising an augmented abundance of at least one isotope of an element selected from the group consisting of lithium, rubidium, and cesium.

14. The method of claim 13, wherein tagging an ink with a tagging composition comprising an augmented abundance of at least one isotope of an element to produce an authentic ink comprises tagging an ink with a tagging composition comprising an augmented abundance of at least one isotope selected from the group consisting of ⁶lithium, ⁷lithium, ⁸⁵rubidium, ⁸⁷rubidium, and ¹³³cesium.

15. The method of claim 11, wherein tagging an ink with a tagging composition comprising an augmented abundance of at least one isotope of an element to produce an authentic ink comprises tagging an ink with a tagging composition comprising an augmented abundance of at least one isotope of an alkaline metal selected from the group consisting of beryllium, magnesium, strontium, and barium.

16. The method of claim 11, wherein tagging an ink with a tagging composition comprising an augmented abundance of at least one isotope of an element to produce an authentic ink comprises tagging an ink with a tagging composition comprising an augmented abundance of at least one isotope of a transition metal selected from the group consisting of manganese, cobalt, nickel, copper, zinc, yttrium, niobium, rhodium, and rhenium.

17. The method of claim 11, wherein tagging an ink with a tagging composition comprising an augmented abundance of at least one isotope of an element to produce an authentic ink comprises tagging an ink with a tagging composition comprising an augmented abundance of at least one isotope of a rare earth element selected from the group consisting of lanthanum, cerium, praseodymium, europium, gadolinium, terbium, and lutetium.

18. The method of claim 11,
wherein tagging an ink with a tagging composition comprising an augmented abundance of at least one isotope of an element comprises tagging an ink with a tagging composition comprising an augmented abundance of at least one isotope of a first element and at least one isotope of a second element, the first and second elements being different from one another, to produce an authentic ink having an artificial abundance of the at least one isotope of the first element and the at least one isotope of the second element each of which exceeds a respective natural abundance thereof in the authentic ink, the at least one isotope of each of the first and second elements being present in the authentic ink in a concentration between 1 and 1000 parts per billion,
wherein detecting an abundance of the at least one isotope in the unidentified ink sample comprises detecting an abundance of the at least one isotope of each of the first and second elements in the unidentified ink sample, and
wherein determining whether the unidentified ink is the authentic ink comprises determining whether the unidentified ink is the authentic ink based upon the detected abundance of the at least one isotope of each of the first and second elements in the unidentified ink sample.

19. The method of claim 18, wherein determining whether the unidentified ink is the authentic ink comprises determining a ratio of the detected abundance of the at least one isotope of the first element in the unidentified ink sample to the respective artificial abundance of the at least one isotope of the first element in the authentic ink and a ratio of the detected abundance of the at least one isotope of the second element in the unidentified ink sample to the respective artificial abundance of the at least one isotope of the second element in the authentic ink, and wherein if the ratio of the detected abundance of one or both of the at least one isotope of the first and second elements to the artificial abundance of the respective isotope in the authentic ink is less than 0.66, the unidentified ink is designated as counterfeit.

20. The method of claim 18, wherein determining whether the unidentified ink is the authentic ink comprises:
determining an unidentified isotope ratio for the unidentified ink sample, the unidentified isotope ratio comprising a ratio of the detected abundance of the at least one isotope of the first element to the detected abundance of the at least one isotope of the second element; and
determining an authentic isotope ratio for the authentic ink sample, the authentic isotope ratio comprising a ratio of the artificial abundance of the at least one isotope of the first element to the artificial abundance of the at least one isotope of the second element,
wherein if the ratio of the unidentified isotope ratio to the authentic isotope ratio is less than 0.66, the unidentified ink is designated as counterfeit.

21. The method of claim 18, wherein tagging an ink with a tagging composition comprising an augmented abundance of at least one isotope of a first element and at least one isotope of second element comprises tagging an ink with a tagging composition comprising an augmented abundance of at least one isotope of a first element and at least one isotope of a second element, each of the first and second elements selected from the group consisting of lithium, rubidium, cesium, beryllium, magnesium, strontium, barium, manganese, cobalt, nickel, copper, zinc, yttrium, niobium, rhodium, rhenium, lanthanum, cerium, praseodymium, europium, gadolinium, terbium, and lutetium.

22. The method of claim 11,
wherein tagging an ink with a tagging composition comprising an augmented abundance of at least one isotope of an element comprises tagging an ink with a tagging composition comprising an augmented abundance of at least two isotopes of an element to produce an authentic ink, the authentic ink having an artificial abundance of the at least two isotopes which exceeds a respective natural abundance thereof in the authentic ink, wherein an authentic isotope ratio of the respective artificial abundances of the at least two isotopes is different from a natural abundance ratio thereof in the authentic ink, and wherein each of the at least two isotopes is present in the authentic ink in a concentration between 1 and 1000 parts per billion,
wherein detecting an abundance of the at least one isotope in the unidentified ink sample comprises detecting an abundance of each of the at least two isotopes in the unidentified ink sample, and
wherein determining whether the unidentified ink is the authentic ink comprises determining whether the unidentified ink is the authentic ink based upon the detected abundances of each of the at least two isotopes in the unidentified ink sample.

23. A method for authenticating an ink, comprising:
obtaining a sample of an unidentified ink;
detecting an abundance of at least one isotope of an element in the unidentified ink sample using inductively coupled plasma mass spectrometry;
comparing the detected abundance to a tagging record which correlates an authentic ink identifier with information regarding an authentic ink, the authentic ink having an artificial abundance of the at least one isotope which exceeds a natural abundance of the at least one isotope in the authentic ink, the at least one isotope being present in the authentic ink in a concentration between 1 and 1000 parts per billion; and
determining whether the unidentified ink is the authentic ink based upon a comparison of the detected abundance of the at least one isotope in the unidentified ink sample and the tagging record.

24. The method of claim 23, wherein determining whether the unidentified ink is the authentic ink comprises determining the ratio of the detected abundance of the at least one isotope in the unidentified ink sample to the artificial abundance of the at least one isotope in the authentic ink indicated in the tagging record, wherein if the ratio of the detected abundance to the artificial abundance is less than 0.66, the unidentified ink is designated as counterfeit.

25. The method of claim 23, wherein detecting an abundance of at least one isotope of an element in the unidentified ink sample comprises detecting an abundance of at least one isotope of an element selected from the group consisting of lithium, rubidium, and cesium.

26. The method of claim 25, wherein detecting an abundance of at least one isotope of an element in the unidentified ink sample comprises detecting an abundance of at least one isotope selected from the group consisting of ⁶lithium, ⁷lithium, ⁸⁵rubidium, ⁸⁷rubidium, and ¹³³cesium.

27. The method of claim 23, wherein detecting an abundance of at least one isotope of an element in the unidentified ink sample comprises detecting an abundance of at least one isotope of an alkaline metal selected from the group consisting of beryllium, magnesium, strontium, and barium.

28. The method of claim 23, wherein in detecting an abundance of at least one isotope of an element comprises detecting an abundance of at least one isotope of rubidium, beryllium, or magnesium, and wherein the at least one rubidium, beryllium, or magnesium isotope is present in the authentic ink in a concentration between 10 and 1000 parts per billion.

29. The method of claim 23, wherein detecting an abundance of at least one isotope of an element in the unidentified ink sample comprises detecting an abundance of at least one isotope of a transition metal selected from the group consisting of manganese, cobalt, nickel, copper, zinc, yttrium, niobium, rhodium, and rhenium.

30. The method of claim 23, wherein detecting an abundance of at least one isotope of an element in the unidentified ink sample comprises detecting an abundance of at least one isotope of a rare earth element selected from the group consisting of lanthanum, cerium praseodymium, europium, gadolinium, terbium, and lutetium.

31. The method of claim 23,
wherein detecting an abundance of at least one isotope of an element in the unidentified ink sample comprises detecting an abundance of at least one isotope of a first element and an abundance of at least one isotope of a second element, the first and second elements being different from one another,
wherein comparing the detected abundance to a tagging record comprises comparing the detected abundance of the at least one isotope of the first element and the detected abundance of the at least one isotope of the second element to the tagging record which correlates the authentic ink identifier with information regarding the authentic ink, the authentic ink having an artificial abundance of the at least one isotope of the first element and an artificial abundance of the at least one isotope of the second element, the respective artificial abundances exceeding a respective natural abundance thereof in the authentic ink, each of the respective artificial abundances of the at least one isotopes of the first and second elements being present in the authentic ink in a concentration between 1 and 1000 parts per billion, and
wherein determining whether the unidentified ink is the authentic ink comprises determining whether the unidentified ink is the authentic ink based upon a comparison of the detected abundance of the at least one isotope of each of the first and second elements in the unidentified ink sample and the tagging record.

32. The method of claim 23,
wherein detecting an abundance of at least one isotope of an element in the unidentified ink sample comprises detecting an abundance of each of at least two isotopes of an element in the unidentified ink sample,
wherein comparing the detected abundance to a tagging record comprises comparing the detected abundance of each of the at least two isotopes of the element in the unidentified ink sample with the tagging record which correlates the authentic ink identifier with information regarding the authentic ink, the authentic ink having an artificial abundance of each of the at least two isotopes of the element, the artificial abundance of each of the at least two isotopes of the element exceeding a respective natural abundance thereof in the authentic ink, and an authentic isotope ratio of the respective artificial abundances of the at least two isotopes being different from a natural abundance ratio of the at least two isotopes, each of the at least two isotopes being present in the authentic ink in a concentration between 1 and 1000 parts per billion, and
wherein determining whether the unidentified ink is the authentic ink comprises determining whether the unidentified ink is the authentic ink based upon a comparison of the detected abundances of each of the at least two isotopes of the element in the unidentified ink sample and the tagging record.

33. A method for tagging a pigment-based ink for identification, comprising:
providing the pigment-based ink; and
mixing the ink with a composition having a trityl-protected thiol additive.

34. The method of claim 33, wherein the trityl-protected additive is S-tritylcysteine or a derivative thereof.

35. A method for authenticating an ink, comprising:
obtaining a sample of an unidentified pigment-based ink;
adding an acid to the pigment-based ink to cause the pigment to coagulate;
removing the coagulated pigment from the ink; and
assaying the coagulated pigment.

36. The method of claim 35, wherein removing the coagulated pigment from the ink comprises filtering or centrifuging the pigment-based ink.

37. The method of claim 35, wherein assaying the coagulated pigment comprises analyzing the coagulated pigment by an Ellman assay and detecting the absorbance of a resulting product from the Ellman assay.
